(19) United States
(12) Patent Application Publication
(10) Pub. No.: US 2024/0158693 A1
(43) Pub. Date: May 16, 2024

(54) Probe for Hydrogen Sulfide Detection, Method for Manufacturing Same, and Composition for Hydrogen Sulfide Detection, Comprising Same

(71) Applicant: Korea Institute of Ocean Science &Technology, Busan (KR)

(72) Inventors: Jong Seok LEE, Busan (KR); Dan-Bi SUNG, Busan (KR); Yeon-Ju LEE, Busan (KR); Jihoon LEE, Busan (KR); Hyi Seung LEE, Busan (KR); Hee Jae SHIN, Busan (KR)

(21) Appl. No.: 18/267,088

(22) PCT Filed: Nov. 11, 2021
(86) PCT No.: PCT/KR2021/016446

§ 371 Date: Jan. 4, 2024

(30) Foreign Application Priority Data
Dec. 15, 2020 (KR) 10-2020-0175242

(51) Int. Cl. C09K 11/06; C09B 57/00; C09B 67/20; G01N 21/64; G01N 33/84

(52) U.S. Cl. CPC C09K 11/06; C09B 57/00; C09B 67/0065; G01N 21/6428; G01N 33/84; C09K 2211/1007; C09K 2211/1014; C09K 2211/1018; G01N 2021/6439

(57) ABSTRACT

The probe for hydrogen sulfide detection according to the present invention is represented by chemical formula 1 below.

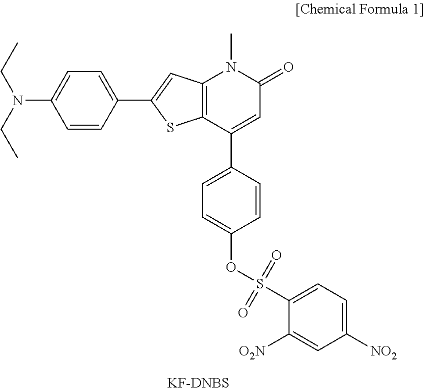 [SMILES: CCN(CC)c1ccc(-c2cc3c(s2)c(-c2ccc(OS(=O)(=O)c4ccc([N+](=O)[O-])cc4[N+](=O)[O-])cc2)cc(=O)n3C)cc1 ; "KF-DNBS"]

### TECHNICAL FIELD

Various embodiments of the present disclosure are drawn to a probe for detection of hydrogen sulfide, a manufacturing method therefor, and a composition including same for detection of hydrogen sulfide. Specifically, various embodiments of the present disclosure pertain to a probe for detection of hydrogen sulfide, which can selectively and conveniently detect hydrogen sulfide in blood, a manufacturing method therefor, and a composition containing same for detection of hydrogen sulfide.

### BACKGROUND ART

Hydrogen sulfide (H₂S) is emerging as a significant endogenous gas mediator, like the well-known nitric oxide (NO) and carbon monoxide (CO). Perturbed synthesis of endogenous H₂S is closely associated with various diseases. Recent studies have shown that abnormal serum levels of H₂S are observed in several physiological disorders such as Alzheimer's disease, hypertension, diabetes, and asthma. Hence, the development of a reliable detection method for H₂S in serum has great importance in pathology. Moreover, fast and real-time monitoring is required considering the rapid metabolism of H₂S in physiological processes.

To date, a variety of analytical techniques such as spectrophotometry, electrochemical assay, and chromatography (including gas, ion-exchange, and variants of high-performance liquid chromatography (HPLC)) have been reported for H₂S detection. Among them, two common methods have been widely used for measuring H₂S levels in serum: a colorimetric method using methylene blue (MB method) and an ion-selective electrode (ISE)-based sulfide anion (S2-)-specific method. Both the methods are performed under harsh chemical conditions and also possess several practical drawbacks, such as tedious sample processing and the requirement of sophisticated instruments.

In contrast, fluorescent small-molecule probes have great potential for real-time monitoring of H₂S in terms of their simplicity, rapid response, and high sensitivity. However, most of them are focused on the fluorescence imaging of H₂S, and it is intricate to be applied for the measurement of H₂S levels in serum samples since they suffer signal interference due to nonspecific binding of the fluorophore with serum proteins (FIG. 1a). To avoid signal interference, which could lead to inaccurate measurement of H₂S in serum, an additional process to remove the large amounts of proteins in serum samples before H₂S measurement is essential. In addition, many thiolysis-based probes are susceptible to interference from other biothiols present at high concentrations in serum, such as cysteine (Cys) and homocysteine (Hcy), which have similar reactivity to H₂S. Thus, it is still necessary to develop a highly selective fluorescent probe that is readily applicable to H₂S quantitation in serum samples.

### DISCLOSURE OF INVENTION

### Technical Problem

With the above problems in mind, the present disclosure is designed and aims to provide a highly selective fluorescent probe for H₂S detection.

### Solution to Problem

The probe for hydrogen sulfide detection is represented by the following Chemical Formula 1:

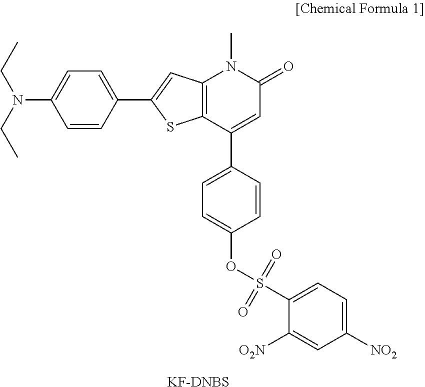 [SMILES: CCN(CC)c1ccc(-c2cc3c(s2)c(-c2ccc(OS(=O)(=O)c4ccc([N+](=O)[O-])cc4[N+](=O)[O-])cc2)cc(=O)n3C)cc1 ; "KF-DNBS"]

A method for manufacturing a probe for hydrogen sulfide detection according to the present disclosure includes the steps of:
preparing a first intermediate (5-(4-(diethylamino)phenyl)-N-methylthiophen-3-amine) (5a);
obtaining a second intermediate (2-(4-(diethylamino)phenyl)-7-(4-methoxyphenyl)-4-methyl-6,7-dihydrothieno[3,2-b]pyridin-5(4H)-one) (6a) by quenching the first intermediate (5a) with water and extraction;
obtaining a third intermediate (6-bromo-2-(4-(diethylamino)phenyl)-7-(4-methoxyphenyl)-4-methylthieno[3,2-b]pyridin-5(4H)-one) (7a) by quenching the second intermediate (6a) with water and extraction;
obtaining a fourth intermediate (2-(4-(Diethylamino)phenyl)-7-(4-methoxyphenyl)-4-methylthieno[3,2-b]pyridin-5(4H)-one) (8a) by quenching the third intermediate (7a) with water and extraction;
obtaining KF (2-(4-(diethylamino)phenyl)-7-(4-hydroxyphenyl)-4-methylthieno[3,2-b]pyridin-5(4H)-one) by quenching the fourth intermediate (8a) with water and extraction; and
obtaining KF-DNBS (4-(2-(4-(diethylamino)phenyl)-4-methyl-5-oxo-4,5-dihydrothieno[3,2-b]pyridin-7-yl)phenyl 2,4-dinitrobenzenesulfonate) by quenching the KF with water and extraction.

The step of obtaining the second intermediate (6a) is characterized by mixing and reacting the first intermediate (5a) with 4-methoxycinnamic acid, benzotriazol-1-yloxy)tris(dimethylamino)phosphonium hexafluorophosphate (BOP) and DIPEA.

The step of obtaining the third intermediate (7a) includes a step of mixing and reacting the second intermediate (6a) with N-bromosuccinimide.

The step of obtaining the fourth intermediate (8a) includes a step of quenching the third intermediate (7a), followed by reaction with an n-butyllithium solution.

The step of obtaining the KF includes the steps of:
quenching the fourth intermediate (8a), followed by reaction with boron tribromide; and
quenching the resulting reaction mixture with water, followed by neutralization.

The step of obtaining the KF-DNBS includes a step of mixing and reacting KF with 2,4-dinitrobenzenesulfonyl chloride and triethylamine.

The composition for hydrogen sulfide detection according to various embodiments of the present disclosure includes: a probe, represented by Chemical Formula 1, for hydrogen sulfide (H₂S) detection; and 2-formyl benzene boronic acid (2-FBBA) as a masking reagent.

### Advantageous Effects of Invention

KF-DNBS, which is the probe for hydrogen sulfide detection of the present disclosure, undergoes the H₂S-induced thiolysis, forming the fluorescent KF-albumin complex that exhibits remarkable fluorescence enhancement. In addition, the introduction of 2-FBBA can improve the selectivity of KF-DNBS to H₂S by blocking the reactivity of Cys and Hcy based on the fast and chemoselective reaction of 2-FBBA with Cys and Hcy.

Furthermore, under optimized sensing conditions, KF-DNBS can be applied to accurately detect spiked H₂S in human serum without the need for any further procedure for the removal of serum proteins.

Therefore, the fluorescent reaction of KF-DNBS can be used as a method for accurately and conveniently measure H₂S levels in serum samples.

### BRIEF DESCRIPTION OF DRAWINGS

FIG. 1 a) is a schematic view illustrating common problems in application of conventional fluorescent probes for H₂S in serum and FIG. 1 b) is a schematic illustration of application of the fluorescent KF-DNBS probe of the present disclosure for facile H₂S detection in serum using the H₂S-triggered cascade formation of the fluorescent KF-albumin complex.

FIG. 2 shows fluorescence spectra of KF and KF-DNBS (each 25 μM, 10% DMSO) in the absence and presence of HSA (100 μM) (λex=420 nm), with a fluorescence image of KF in the absence and presence of HSA under handheld UV lamp (365 nm) illumination (inset).

FIG. 3 a) shows changes of fluorescence intensity of KF-DNBS (25 μM, 10% DMSO) with HSA (100 μM) upon addition of the individual biothiols H₂S (100 μM), Cys (250 μM), Hcy (100 μM), and GSH (10 μM) in the absence of 2-FBBA and FIG. 3 b) shows changes of fluorescence intensity in the presence of 2-FBBA (2 mM).

FIG. 4 a) shows fluorescence spectral changes of KF-DNBS (25 μM, 10% DMSO) with HSA (100 μM) in the absence and presence of H₂S (100 μM), FIG. 4 b) is a plot of fluorescence intensity at 500 nm of KF-DNBS (25 μM, 10% DMSO) with HSA (100 μM) versus different concentrations of H₂S (5-250 μM) in SPB (pH 7.4, 20 mM) containing 2-FBBA (2 mM), and FIG. 4 c) is a graph of fluorescence intensity at 500 nm of KF-DNBS (25 μM, 10% DMSO) with HSA (100 μM) in the absence and presence of H₂S (100 μM) in various buffer conditions (pH 5-9, 20 mM).

FIG. 5 is a graph of fluorescence intensity at 500 nm of KF-DNBS (25 μM, 10% DMSO) with HSA (100 μM) in the presence of various analytes:
(1: blank, 2: H₂S (100 μM), 3: Cys (250 μM), 4: Hcy (100 μM), 5: GSH (10 μM), 6: HSO₄⁻ (100 μM), 7: SO₄²⁻ (100 μM), 8: SO₃²⁻ (100 μM), 9: S₂O₃²⁻ (100 μM), 10: SCN⁻ (100 μM), 11: CN⁻ (100 μM), 12: F⁻ (100 μM), 13: Br⁻ (100 μM), 14: NO₃⁻ (100 μM), 15: NO₂⁻ (100 μM), 16: HCO₃⁻ (100 μM), 17: CH₃CO₂⁻ (100 μM), 18: H₂O₂ (100 μM), 19: ClO⁻ (100 μM) in SPB (pH 7.4, 20 mM))

### BEST MODE FOR CARRYING OUT THE INVENTION

Hereinafter, various embodiments of this document will be described with reference to the accompanying drawings. However, it should be understood that technology described in this document is not limited to a specific embodiment and includes various modifications, equivalents, and/or alternatives of an embodiment of this document.

A probe for hydrogen sulfide detection according to the present disclosure is represented by the following Chemical Formula:

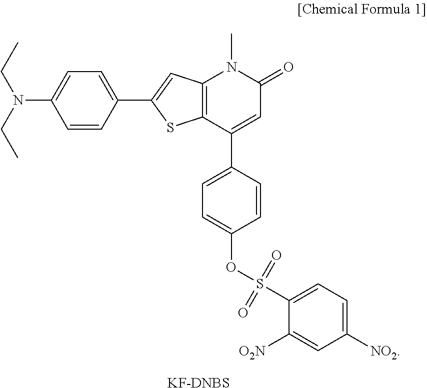 [SMILES: CCN(CC)c1ccc(-c2cc3c(s2)c(-c2ccc(OS(=O)(=O)c4ccc([N+](=O)[O-])cc4[N+](=O)[O-])cc2)cc(=O)n3C)cc1 ; "KF-DNBS"]

The probe for hydrogen sulfide detection according to the present disclosure is 4-(2-(4-(diethylamino)phenyl)-4-methyl-5-oxo-4,5-dihydrothieno[3,2-b]pyridin-7-yl)phenyl 2,4-dinitrobenzenesulfonate (KF-DNBS).

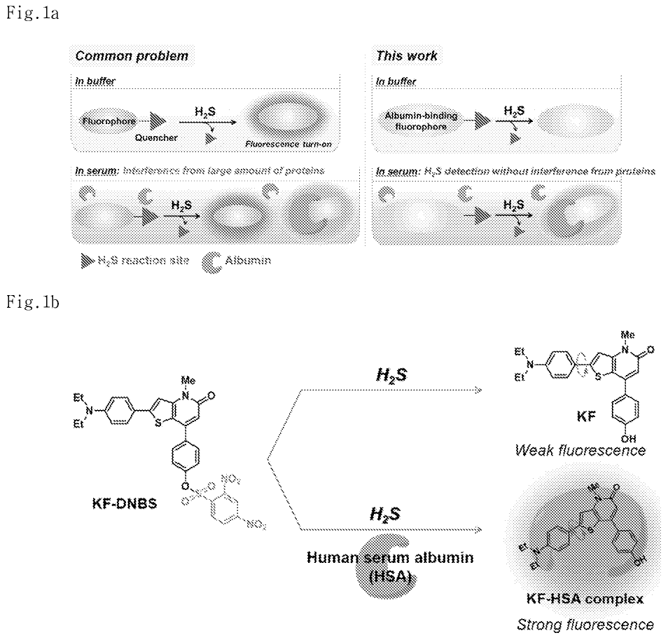

With reference to FIG. 1b), the fluorescence of KF greatly depends on its specific binding to HSA, and the DNBS group is cleaved by H₂S. That is, in response to H₂S, the DNBS group in KF-DNBS is cleaved by thiolysis, thus releasing KF which then immediately combines with albumin, resulting in significant fluorescence enhancement. Based on this H₂S-triggered cascade formation of the fluorescent KF-albumin complex, KF-DNBS can be used in the quantitative detection of H₂S in physiological conditions and can detect H₂S without additional processing for the removal of serum proteins

A method for manufacturing the probe for hydrogen sulfide according to the present disclosure may be carried out as illustrated in the following reaction scheme:

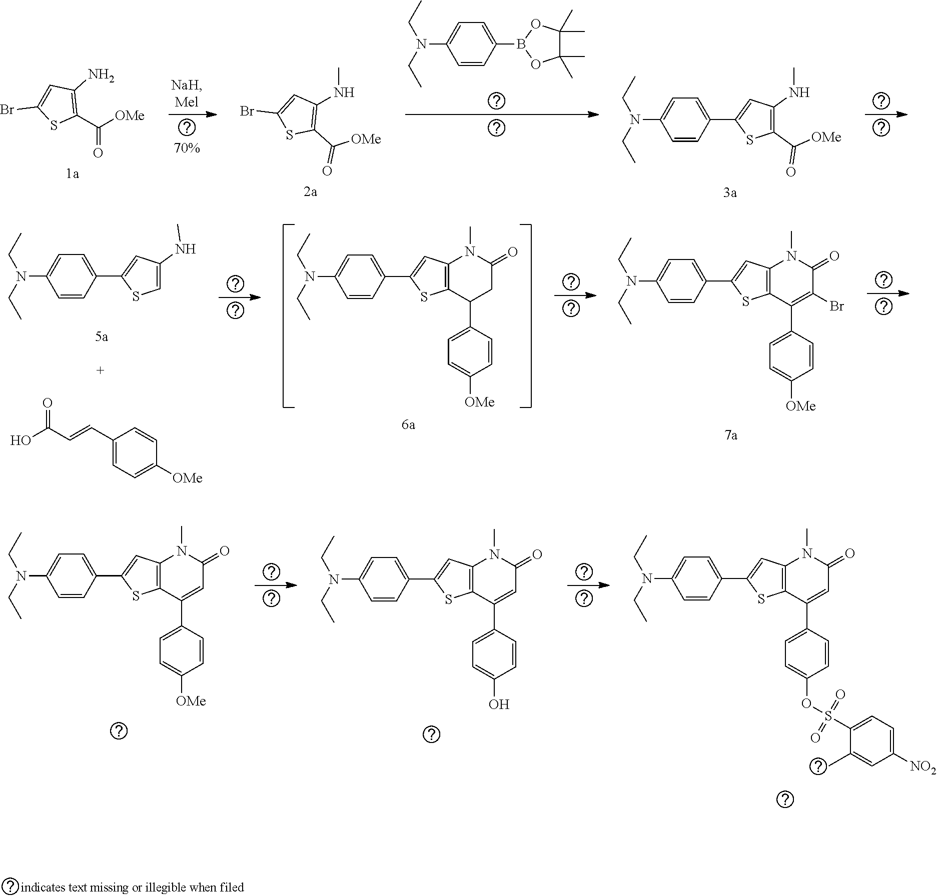

In detail, the method includes the steps of: preparing a first intermediate (5-(4-(diethylamino)phenyl)-N-methylthiophen-3-amine) (5a); obtaining a second intermediate (2-(4-(diethylamino)phenyl)-7-(4-methoxyphenyl)-4-methyl-6,7-dihydrothieno[3,2-b]pyridin-5(4H)-one) (6a) by quenching the first intermediate (5a) with water and extraction; obtaining a third intermediate (6-bromo-2-(4-(diethylamino)phenyl)-7-(4-methoxyphenyl)-4-methylthieno[3,2-b]pyridin-5(4H)-one) (7a) by quenching the second intermediate (6a) with water and extraction; obtaining a fourth intermediate (2-(4-(diethylamino)phenyl)-7-(4-methoxyphenyl)-4-methylthieno[3,2-b]pyridin-5(4H)-one) (8a) by quenching the third intermediate (7a) with water and extraction; obtaining KF (2-(4-(diethylamino)phenyl)-7-(4-hydroxyphenyl)-4-methylthieno[3,2-b]pyridin-5(4H)-one) by quenching the fourth intermediate (8a) with water and extraction; and obtaining KF-DNBS (4-(2-(4-(diethylamino)phenyl)-4-methyl-5-oxo-4,5-dihydrothieno[3,2-b]pyridin-7-yl)phenyl 2,4-dinitrobenzenesulfonate) by quenching the KF with water and extraction.

The step of obtaining the first intermediate (5a) includes the steps of synthesizing compound 2a; synthesizing compound 3a from compound 2a; and synthesizing compound 5a from compound 3a.

First, in the step of synthesizing compound 2a, 3-amino-5-bromothiophene-2-carboxylate (1a) is added and reacted with NaH and methyl iodide, followed by extraction to afford methyl 5-bromo-3-(methylamino)thiophene-2-carboxylate (2a).

In the step of synthesizing compound 3a, compound 2a is added and reacted with Pd(PPh₃)4, N,N-diethyl-4-(4,4,5,5-tetramethyl-1,3,2-dioxaborolan-2-yl)aniline, K₂CO₃, and H₂O, followed by extraction to afford methyl 5-(4-(diethylamino)phenyl)-3-(methylamino)thiophene-2-carboxylate (3a).

In the step of synthesizing compound 5a, compound 3a is added and reacted with KOH, followed by extraction to afford 5-(4-(diethylamino)phenyl)-N-methylthiophen-3-amine (5a).

The step of obtaining the second intermediate (6a) may be carried out by mixing and reacting the first intermediate (5a) with 4-methoxycinnamic acid, benzotriazol-1-yloxy)tris(dimethylamino)phosphonium hexafluorophosphate (BOP) with DIPEA.

The step of obtaining the third intermediate (7a) includes the step of mixing and reacting the second intermediate (6a) with N-bromosuccinimide.

The step of obtaining the fourth intermediate (8a) includes the step of cooling the third intermediate (7a) and reacting same with an n-butyllithium solution.

The step of obtaining the KF includes the steps of: quenching the fourth intermediate (8a) and reacting the same with boron tribromide; and quenching the resulting reaction mixture with quenching, followed by neutralization.

The step of obtaining the KF-DNBS includes a step of mixing and reacting KF with 2,4-dinitrobenzenesulfonyl chloride and triethylamine.

The composition for hydrogen sulfide detection according to various embodiments of the present disclosure includes: a probe, represented by Chemical Formula 1, for hydrogen sulfide (H₂S) detection; and 2-formyl benzene boronic acid (2-FBBA) as a masking reagent.

The reactivity of Cys and Hcy is blocked by 2-FBBA, thereby improving the selectivity of KF-DNBS to H₂S.

Below, a detailed description will be given of the present disclosure through the following Examples. A better understanding of the present disclosure may be obtained through the following examples, which are set forth to illustrate, but are not to be construed to limit the present disclosure.

### <EXAMPLE 1> SYNTHESIS OF METHYL 5-BROMO-3-(METHYLAMINO)THIOPHENE-2-CARBOXYLATE (2A)

To a solution of methyl 3-amino-5-bromothiophene-2-carboxylate (1.00 g, 4.24 mmol, 1.0 equiv.) in DMF (40 mL) was added NaH (60% in mineral oil dispersion, 237 mg, 11.9 mmol, 1.4 equiv.) at 0° C. After being stirred for 10 minutes, the solution was added with methyl iodide (343 μL, 5.51 mmol, 1.3 equiv.) and warmed to room temperature. The reaction mixture was stirred at room temperature for 16 hours and quenched with water (50 mL), followed by three rounds of extraction with EtOAc (50 mL). The organic layer was dried over Na₂SO₄, filtered, and evaporated in a vacuum. The crude product was purified by flash column chromatography (hexane/EtOAc=30/1, v/v) on silica to afford product 2a as a white solid.

NMR data for compound 2a are as follows.

¹H NMR (600 MHz, CDCl₃) δ6.67 (br s, 1H), 6.63 (s, 1H), 3.78 (s, 3H), 2.93 (d, J=5.5 Hz, 3H);

¹³C NMR (150 MHz, CDCl₃) δ164.4, 156.5, 121.8, 119.5, 99.2, 51.3, 31.7.

### <EXAMPLE 2> SYNTHESIS OF METHYL 5-(4-(DIETHYLAMINO)PHENYL)-3-(METHYLAMINO)THIOPHENE-2-CARBOXYLATE (3A)

To a solution of methyl 5-bromo-3-(methylamino)thiophene-2-carboxylate (2a) (1.04 g, 4.14 mmol, 1.0 equiv.) in 1,2-dimethoxyethane (13.8 mL, 0.3 M) were added Pd(PPh₃)4 (239.4 mg, 0.2072 mmol, 0.05 equiv.), N,N-diethyl-4-(4,4,5,5-tetramethyl-1,3,2-dioxaborolan-2-yl)aniline (1.37 g, 4.97 mmol, 1.2 equiv.), K₂CO₃ (1.72 g, 12.43 mmol, 3.0 equiv.) and H₂O (0.3 mL). The mixture was heated at 80° C. and stirred for 12 hours. After completion of the reaction, the reaction mixture was filtered through celite and subjected to three rounds of extraction with H₂O/EtOAc. The organic layer thus formed was dried over Na₂SO₄ and filtered before evaporation in vacuum. The crude was purified by flash column chromatography using a mixture of n-hexane/EtOAc (5:1) on silica to afford product 3a as a yellow solid (1.26 g, 3.95 mmol, 96%).

NMR data for compound 3a are as follows.

¹H NMR (600 MHz, CDCl₃) δ7.50 (d, J=9.0Hz, 2H), 6.70 (s, 1H), 6.65 (d, J=9.0Hz, 2H), 3.81 (s, 3H), 3.33 (q, J=7.0Hz, 4H), 3.02 (d, J=4.8Hz, 3H), 1.19 (t, J=7.2Hz, 6H); ¹³C {¹H} NMR (150 MHz, CDCl₃) δ165.3, 158.0, 151.4, 148.3, 127.3, 120.5, 111.4, 108.7, 94.9, 50.9, 44.4, 31.6, 12.6;

### <EXAMPLE 3> SYNTHESIS OF 5-(4-(DIMETHYLAMINO)PHENYL)-N-METHYLTHIOPHEN-3-AMINE (5A)

To a solution of methyl 5-(4-(diethylamino)phenyl)-3-(methylamino)thiophene-2-carboxylate (3a) (1.20 g, 4.13 mmol, 1.0 equiv.) in ethanol (4 mL, 0.25 M) was added 1N KOH (2 mL). The mixture was stirred for 2 hours while being stirred at 70° C. After completion of the reaction, the solvent was evaporated. Then, the crude product was reacted with a silica gel without further purification. In this regard, the crude product was added to a solution of silica gel (750 mg, 500 wt % of the substrate) in a mixture of EtOAc (2 mL) and MeOH (2 mL) (1:1) and stirred at room temperature for 1 hour. After the silica gel was filtered out, the organic layer was dried in a vacuum. The residue was purified by flash column chromatography (hexane/EtOAc=3/1, v/v) on silica to afford compound 5a as a reddish brown solid (600 mg, 2.58 mmol, 62%).

NMR data for compound 5a are as follows.

¹H NMR (600 MHz, CDCl₃) δ7.44 (d, J=9.0 Hz, 2H), 6.71-6.70 (m, 3H), 5.81 (s, 1H), 2.98 (s, 6H), 2.84 (s, 3H) ; ¹³C NMR (150 MHz, CDCl₃) δ150.2, 150.1, 144.5, 126.6, 123.3, 114.0, 112.6, 93.0, 40.6, 32.8; LRMS (APCI): m/z calcd for C₁₃H₁₇N₂S [M+H]⁺233.11, found 232.80.

### <EXAMPLE 4> SYNTHESIS OF 6-BROMO-2-(4-(DIETHYLAMINO)PHENYL)-7-(4-METHOXYPHENYL)-4-METHYLTHIENO[3,2-B]PYRIDIN-5(4H)-ONE (7A)

To a solution of 5-(4-(diethylamino)phenyl)-N-methylthiophen-3-amine (5a) (150 mg, 0.5760 mmol, 1.0 equiv.) in DMF (2.3 mL, 0.25M) was added 4-methoxycinnamic acid (225.8 mg, 1.267 mmol, 2.2 equiv.), benzotriazol-1-yloxy)tris(dimethylamino)phosphonium hexafluorophosphate (BOP, 560 mg, 1.267 mmol, 2.2 equiv.), and DIPEA (0.51 mL, 2.880 mmol, 5.0 equiv.) at room temperature. The mixture was stirred at room temperature for 5 hours. The reaction mixture was quenched with H₂O before three rounds of extraction with ethyl acetate (EtOAc). The organic layer thus formed was dried over Na₂SO₄ and filtered, followed by evaporation in a vacuum. 2-(4-(Diethylamino)phenyl)-7-(4-methoxyphenyl)-4-methyl-6,7-dihydrothieno[3,2-b]pyridin-5(4H)-one (6a) was obtained. The crude product 6a was used without further purification.

To a solution of 6a (188.0 mg, 0.4813 mmol, 1.0 equiv.) in CH₂Cl₂ (4.8 mL, 0.1 M) was added N-bromosuccinimide (128.5 mg, 0.7221 mmol, 1.5 equiv.). The mixture was stirred at room temperature for 1 hour. The reaction mixture was quenched with H₂O before three rounds of extraction with ethyl acetate (EtOAc). The organic layer thus formed was dried over Na₂SO₄ and filtered, followed by evaporation in a vacuum. The crude thus obtained was purified by flash column chromatography using a mixture of n-hexane:EtOAc (1:1) on silica to afford compound 7a as a yellow solid (22.4 mg, 0.0532 mmol, 48%, over two steps); m.p: 212-214° C.;

NMR data for compound 7a are as follows.

¹H NMR (600 MHz, CDCl₃) δ7.45-7.41 (m, 4H), 7.05 (s, 1H), 7.03 (d, J=8.4 Hz, 2H), 6.62 (d, J=9.0 Hz, 2H), 3.88 (s, 3H), 3.82 (s, 3H), 3.38 (q, J=7.0 Hz, 4H), 1.18 (t, J=6.9 Hz, 6H); ¹³C{¹H} NMR (150 MHz, CDCl₃) δ160.2, 159.3, 150.8, 148.6, 146.4, 143.2, 130.2, 129.9, 127.3, 119.9, 118.4, 114.1, 111.6, 111.3, 108.7, 55.4, 44.5, 33.6, 12.7; HRMS (EI) : m/z calcd for C₂₅H₂₅BrN₂NaO₂S [M+Na]+ 519.0718, found 519.0719.

### <EXAMPLE 5> SYNTHESIS OF 2-(4-(DIETHYLAMINO)PHENYL)-7-(4-METHOXYPHENYL)-4-METHYLTHIENO[3,2-B]PYRIDIN-5(4H)-ONE (8A)

A solution of 7a (36.1 mg, 0.0726 mmol, 1.0 equiv.) in CH₂Cl₂ (0.73 mL, 0.1 M) was cooled down to −78° C. Next, an n-butyllithium solution (2.0 M in cyclohexane, 75 μL, 0.109 mmol, 1.5 equiv.) was slowly added and stirred at −78° C. for 1.5 hours. The reaction mixture was quenched with H₂O before three rounds of extraction with CH₂Cl₂. The organic layer thus formed was dried over Na₂SO₄ and filtered, followed by evaporation in a vacuum. The crude was purified by flash column chromatography using a mixture of n-hexane:EtOAc (1:1) on silica to afford 8a as a yellow solid (15.8 mg, 0.0377 mmol, 52%); m.p: 190-192° C.;

NMR data for compound 8a are as follows.

¹H NMR (600 MHz, CDCl₃) δ7.64 (d, J=8.4 Hz, 2H), 7.51 (d, J=8.4 Hz, 2H), 7.10 (s, 1H), 7.02 (d, J=8.4 Hz, 2H), 6.67 (d, J=8.4 Hz, 2H), 6.51 (s, 1H), 3.88 (s, 3H), 3.76 (s, 3H), 3.40 (q, J=7.2 Hz, 4H), 1.19 (t, J=6.9 Hz, 6H); ¹³C{¹H} NMR (150 MHz, CDCl₃) δ163.0, 160.7, 150.1, 148.5, 146.7, 145.3, 130.1, 129.1, 127.4, 120.3, 116.4, 114.5, 113.3, 111.7, 109.2, 55.5, 44.6, 31.9, 12.7; HRMS (EI): m/z calcd for C₂₅H₂₆N₂O₂S [M]⁺ 418.1715, found 418.1711.

### <EXAMPLE 6> SYNTHESIS OF 2-(4-(DIETHYLAMINO)PHENYL)-7-(4-HYDROXYPHENYL)-4-METHYLTHIENO[3,2-B]PYRIDIN-5(4H)-ONE (KF)

A solution of 8a (109.8 mg, 0.2623 mmol, 1.0 equiv.) in CH₂Cl₂ (2.6 mL, 0.1 M) was cooled down to −78° C. After cooling, drops of a solution of boron tribromide in CH₂Cl₂ (3.94 mL, 3.935 mmol, 15.0 equiv.) were slowly added to the solution. Next, the mixture was warmed up to room temperature and stirred for 12 hours. After being cooled down to −78° C., the reaction mixture was quenched with iced water and neutralized with NaHCO3. Extraction with CH₂Cl₂ was performed three times. The organic layer thus formed was dried over Na₂SO₄ and filtered before evaporation in a vacuum. The crude was purified by flash column chromatography using a mixture of n-hexane:EtOAc (1:1 to 100% EtOAc) on silica to afford KF as a yellow solid (83.0 mg, 0.2052 mmol, 78%); m.p: 250-251° C.;

NMR data for compound KF are as follows.

¹H NMR (600 MHz, DMSO-d₆) δ9.94 (s, 1H), 7.60-7.58 (m, 3H), 7.56 (d, J=9.0 Hz, 2H), 6.93 (d, J=8.4 Hz, 2H), 6.70 (d, J=9.0 Hz, 2H), 6.27 (s, 1H), 3.65 (s, 3H), 3.38 (q, J=7.0 Hz, 4H), 1.11 (t, J=7.2 Hz, 6H); ¹³C{¹H} NMR (150 MHz, DMSO-d₆) δ161.4, 158.8, 148.7, 148.0, 145.9, 145.5, 128.8, 127.6, 127.0, 119.2, 115.8, 114.0, 111.8, 111.4, 110.4, 43.7, 31.4, 12.4; HRMS (EI): m/z calcd for C₂₄H₂₄N₂O₂S [M]⁺ 404.1558, found 404.1555.

### <EXAMPLE 7> SYNTHESIS OF 4-(2-(4-(DIETHYLAMINO)PHENYL)-4-METHYL-5-OXO-4,5-DIHYDROTHIENO[3,2-B]PYRIDIN-7-YL)PHENYL 2,4-DINITROBENZENESULFONATE (KF-DNBS)

To a solution of KF (106.5 mg, 0.2633 mmol, 1.0 equiv.) in CH₂Cl₂ (2.6 mL, 0.1M) were added 2,4-dinitrobenzenesulfonyl chloride (119.3 mg, 0.4476 mmol, 1.7 equiv.) and triethylamine (0.13 mL, 1.250 mmol, 4.7 equiv.). The mixture was stirred at room temperature for 16 hours. The reaction mixture was quenched with iced water before three rounds of extraction with CH₂Cl₂. The organic layer thus formed was dried over Na₂SO₄ and filtered, followed by evaporation in a vacuum. The crude was purified by flash column chromatography using a mixture of n-hexane:EtOAc (1:1 to 1:5) on silica to afford KF-DNBS as a dark brown solid (108.6 mg, 0.1711 mmol, 65%, conversion:82%, borsm yield: 79%); m.p: 96-98° C.;

¹H NMR (600 MHz, CDCl₃) δ8.67 (s, H), 8.51 (d, J=8.4 Hz, 2H), 8.25 (d, J=8.4 Hz, 2H), 7.67 (d, J=7.8 Hz, 2H), 7.46 (d, J=7.2 Hz, 2H), 7.34 (d, J=8.4 Hz, 2H), 7.09 (s, 1H), 6.65 (br s, 2H), 6.44 (s, 1H), 3.73 (s, 3H), 3.41 (q, J=7.2 Hz, 4H), 1.12 (t, J=6.9 Hz, 6H); ¹³C{¹H} NMR (150 MHz, CDCl₃) δ162.6, 151.1, 150.6, 149.4, 149.1, 148.7, 145.6, 137.7, 134.0, 133.6, 129.7, 127.4, 126.7, 122.7, 120.5, 119.7, 115.4, 114.1, 111.6, 109.1, 44.5, 31.9, 12.6; HRMS (ESI): m/z calcd for C₃₀H₂₇N₄O₈S₂ [M+H]⁺ 635.1270, found 635.1262

### <EXPERIMENTAL EXAMPLE 1> COMPARISON OF FLUORESCENCE CHANGE OF KF AND KF-DNBS WITH HSA

Stock solutions of KF and KF-DNBS were prepared in DMSO, and a stock solution of HSA was prepared in distilled water. Blanks, each containing only KF or KF-DNBS (25 μM, 10% DMSO), and samples, each containing KF or KF-DNBS (25 μM, 10% DMSO) with HSA (100 μM) in sodium phosphate buffer (SPB, pH 7.4, 20 mM), were prepared. Fluorescence spectra were then recorded using the fluorescence spectrophotometer under excitation at 420 nm.

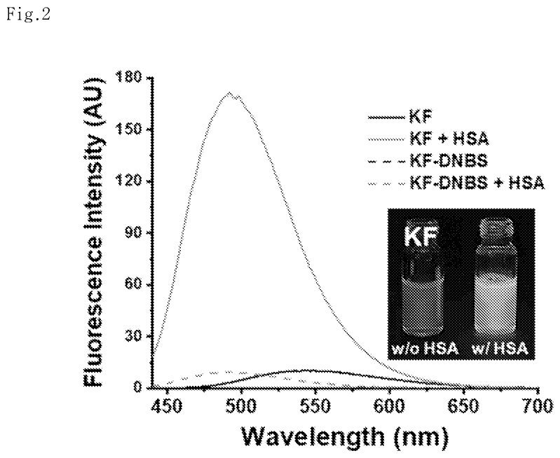

As a result, referring to FIG. 2, the fluorescence intensity of KF shifted slightly from 550 to 500 by addition of HSA and increased linearly with increasing HSA concentration in the range of 5-50 μM. In contrast to KF, KF-DNBS showed weak fluorescence in the presence or absence of HSA. Based on these results, it was envisioned that KF-DNBS with HSA could be utilized as a reaction-based fluorescent probe for H₂S using the principle of H₂S-triggered cascade formation of the fluorescent KF-HSA complex (Φ=0.546).

### <EXPERIMENTAL EXAMPLE 2> OPTIMIZATION OF SENSING CONDITIONS FOR H₂S DETECTION

The samples containing HSA (100 μM) and each biothiol (H₂S 100 μM, Cys 250 μM, Hcy 100 μM, and GSH 10 μM) with and without 2-formyl benzene boronic acid (2-FBBA) in SPB (pH 7.4, 20 mM) were incubated for 15 minutes at 25° C. KF-DNBS (25 μM, 10% DMSO) was added to the samples followed by the measurement of fluorescence spectra under excitation at 420 nm at 37° C.

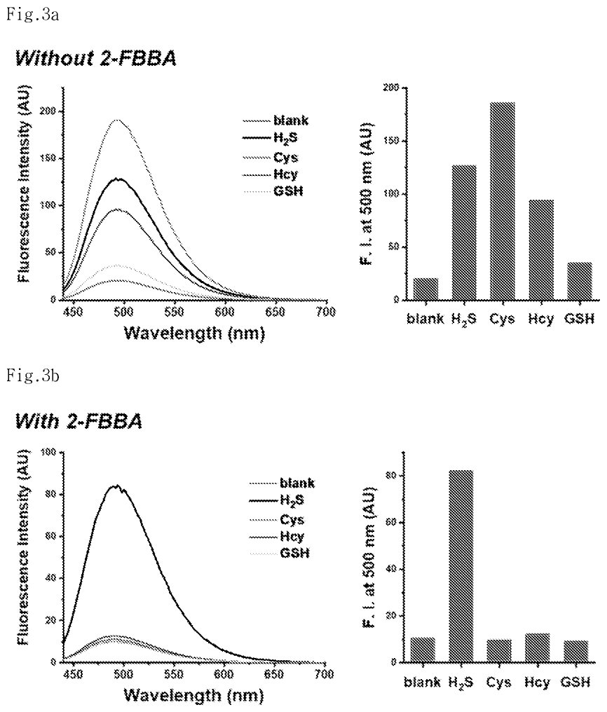

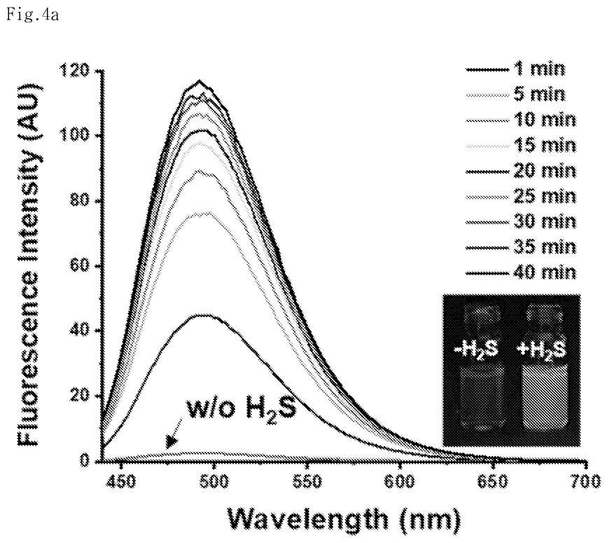

The 2,4-dinitrosulfonyl unit including DNBS has been the most frequently used H₂S recognition unit in H₂S-reactive fluorescent probes. However, these probes usually possessed moderate selectivity because of interference from other biothiols, such as Cys, Hcy, and GSH. Thus, it is desirable to establish the optimal sensing conditions for highly selective H₂S detection by KF-DNBS, especially for reliable application in serum samples containing high concentrations of Cys and Hcy. First, evaluation was made of the relative reactivity of H₂S and other biothiols, including Cys, Hcy, and GSH, to KF-DNBS, considering their approximate concentrations in human serum ([H₂S]=100 μM, [Cys]=250 μM, [Hcy]=100 μM, and [GSH]=10 μM). As shown in FIG. 3a, when H₂S was added to the sample solution containing KF-DNBS with HSA in SPB (pH 7.4), the fluorescence intensity at 500 nm increased significantly as expected. However, Cys and Hcy also induced a noticeable fluorescence change.

To solve this problem, a masking reagent, which can block the nucleophilic reactivity of Cys and Hcy selectively by the formation of a stable covalent bond, was introduced. Since the cyclization reaction between aldehyde groups and Cys or Hcy has been widely used in selective probe molecule design, simple aldehydes were contemplated, and 2-formyl benzene boronic acid (2-FBBA) was chosen as a potential masking reagent. 2-FBBA is a reagent used in facile and selective bioconjugation of N-terminal Cys in proteins at neutral pH. It enables very rapid formation of a stable thiazolidino boronate complex with the boronic acid moiety via a B—N dative bond.

With reference to FIG. 3 b), the capability of 2-FBBA as a masking reagent for selective H₂S detection using KF-DNBS was investigated. KF-DNBS with HSA in the presence of 2-FBBA showed superior selectivity to H₂S over other biothiols. 2-FBBA was able to completely block the reactivity of Cys and Hcy upon KF-DNBS. These results showed, for the first time, that 2-FBBA could be used as an effective masking reagent for Cys and Hcy in thiolysis-based H₂S probes.

### <EXPERIMENTAL EXAMPLE 3> SENSING BEHAVIOR OF KF-DNBS WITH HSA AS SELECTIVE DETECTION H₂S PROBE

Fluorescence spectra of KF-DNBS (25 μM, 10% DMSO) with HSA (100 μM) containing various concentrations of H₂S (0, 5, 10, 20, 40, 60, 80, 100, 150, and 250 μM) in SPB (pH 7.4, 20 mM) were recorded under excitation at 420 nm for 40 minutes at 5-min intervals at 37° C. The experiment was carried out in triplicate. The limit of detection (LOD) was calculated using 3σ/slope based on the titration experiment, in which σ was the standard deviation of the blank measurements and the slope value was obtained from a plot of the fluorescence intensity versus H₂S concentration.

An additional experiment was conducted by using KF-DNBS as a H₂S probe under the following conditions: 25 μM KF-DNBS, 100 μM HAS, and 2-FBBA (2 mM) in SPB (pH 7.4, 20 mM).

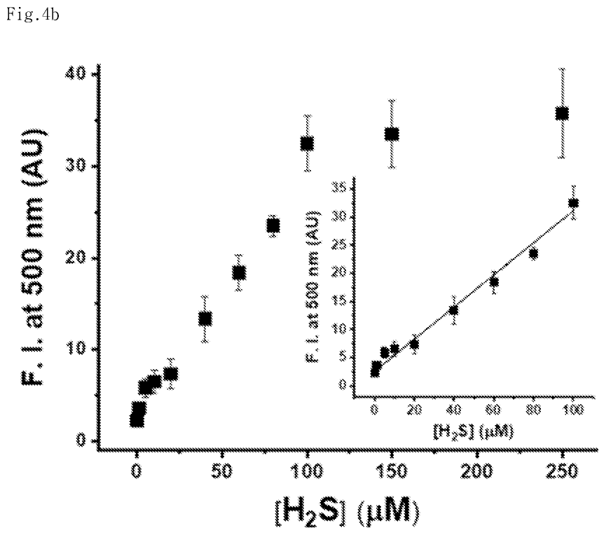

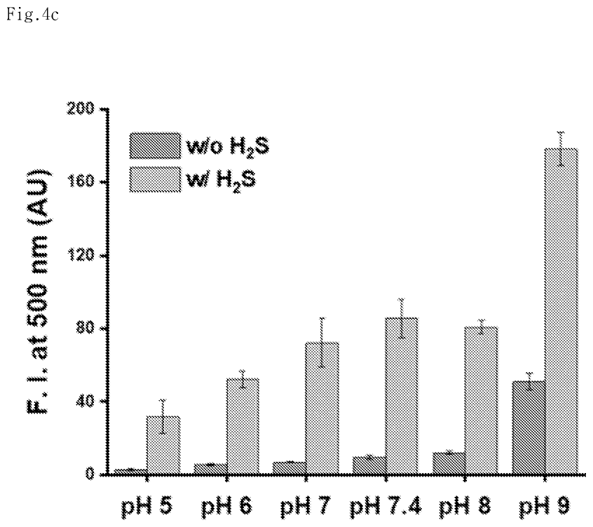

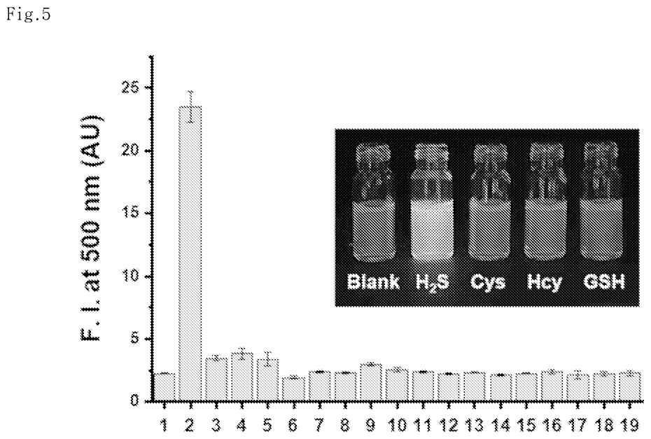

With reference to FIG. 4 a), when H₂S was added to the sensing system containing KF-DNBS with HSA and the masking reagent 2-FBBA in SPB, a great improvement of fluorescence intensity at 500 nm appeared immediately, and the intensity reached the maximum value within 20 minutes. As shown in FIG. 4 b), the fluorescence intensity of KF-DNBS with HSA increased linearly with increasing H₂S concentration (5-100 μM). The detection limit (3σ/slope) was determined to be 3.2 μM, which is reliable for the measurement of serum H₂S levels. That is to say, the probe can respond to a very low concentration of H₂S (LOD=3.2 μM). In addition, the fluorescence change of the sample solution could be monitored with the naked eye, and referring to FIG. 4 c), it was confirmed that KF-DNBS with HSA worked well in a broad pH range from pH 5 to pH 9

### <EXPERIMENTAL EXAMPLE 4> SELECTIVITY OF KF-DNBS TOWARD H₂S OVER OTHER BIOLOGICAL ANALYTES

Next, the selectivity of KF-DNBS with HSA toward H₂S was investigated using various biologically relevant species, including biothiols (Cys, Hcy, and GSH), reactive sulfur species (RSS), and reactive oxygen species (ROS) (HSO₄⁻, SO₄²⁻, SO₃²⁻, S₂O₃²⁻, SCN⁻, H₂O₂, and ClO⁻), and anions (CN⁻, F⁻, Br⁻, NO₃⁻, NO₂⁻, HCO₃⁻, and CH₃CO₂⁻).

A blank containing no analyte and a sample containing each analyte (H₂S 100 μM, Cys 250 μM, Hcy 100 μM, GSH 10 μM, HSO₄⁻ 100 μM, SO₄²⁻ 100 μM, SO₃²⁻ 100 μM, S₂O₃²⁻ 100 μM, SCN⁻ 100 μM, CN⁻ 100 μM, F⁻ 100 μM, Br⁻ 100 μM, NO₃⁻ 100 μM, NO₂⁻ 100 μM, HCO₃⁻ 100 μM, CH₃CO₂⁻ 100 μM, H₂O₂ 100 μM, ClO⁻ 100 μM) were prepared followed by the addition of HSA (100 μM) and 2-FBBA (2 mM) to SPB (pH 7.4, 20 mM). After incubation for 15 minutes at 25° C., KF-DNBS (25 μM, 10% DMSO) was added to each sample, and then fluorescence intensity at 500 nm was recorded using the fluorescence spectrophotometer under excitation at 420 nm at 37° C. The experiment was conducted in triplicate.

As shown in FIG. 5, remarkable fluorescence enhancement was observed only for H₂S. The specific response to H₂S suffered no interference by other analytes, and the selectivity for H₂S over other biothiols was observed with the naked eye. This result demonstrated that KF-DNBS with HSA and the masking reagent 2-FBBA exhibits a high selectivity toward H₂S and works well in complex serum samples containing many biothiols and other reactive species.

### <EXPERIMENTAL EXAMPLE 5> QUANTITATIVE DETECTION OF H₂S IN HUMAN SERUM

To explore the potential applicability of KF-DNBS to facile detection of H₂S in serum, the fluorescence response of KF-DNBS to H₂S-spiked human serum samples was investigated. Human serum (purchased from Sigma Aldrich) was spiked with different concentrations of H₂S (25, 50, 100, and 150 μM). The serum samples, without any pretreatment, were directly added to a solution of 2-FBBA in SPB. Since it has been reported that the concentration of HSA in human serum ranges from 550 to 800 μM, there was no need to add HSA to the sample solutions. After addition of KF-DNBS, the change in fluorescence intensity of the sample solution was measured. Based on the calibration curve obtained from the plot of the initial rate of fluorescence change for 10 minutes versus the concentration of H₂S using KF-DNBS with HSA, the spiked H₂S level in HSA could be determined, and the recovery ranged from 95 to 109%, as shown in Table 1, below.

TABLE

| | Added | Found | Recovery | RSDa |
|---|---|---|---|---|
| | (μM) | (μM) | (%) | (%) |
| Human | 25 | 26 | 106 | 13 |
| serum | 50 | 51 | 101 | 10 |
| | 100 | 95 | 95 | 11 |
| | 150 | 163 | 109 | 12 |

*Relative standard deviation

This result showed that the fluorescence response of KF-DNBS to spiked H₂S was unaffected by the various analytes present in human serum, including high concentrations of biothiols as well as proteins, even though no additional process was performed prior to sample measurement.

It is therefore understood that the fluorescence response of KF-DNBS can be utilized as an accurate and facile method for measuring H₂S levels in serum samples.

The features, structures, effects and the like described in the foregoing embodiments are included in at least one embodiment of the present disclosure and are not necessarily limited to one embodiment. Further, the features, structures, effects, and the like illustrated in the embodiments may be combined or modified in other embodiments by those skilled in the art to which the embodiments belong. Therefore, it should be understood that the present disclosure is not limited to these combinations and modifications.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

1. A probe for hydrogen sulfide detection, represented by the following Chemical Formula 1:

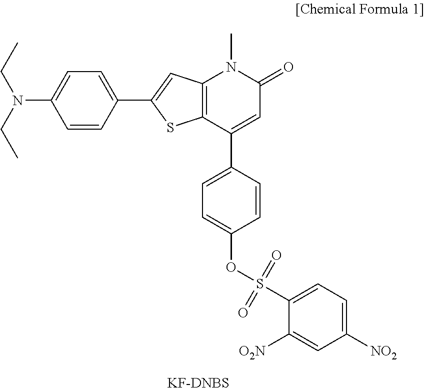 [SMILES: CCN(CC)c1ccc(-c2cc3c(s2)c(-c2ccc(OS(=O)(=O)c4ccc([N+](=O)[O-])cc4[N+](=O)[O-])cc2)cc(=O)n3C)cc1 ; "KF-DNBS"]

2. A method for manufacturing a probe for hydrogen sulfide detection, the method comprising the steps of:
preparing a first intermediate (5-(4-(diethylamino)phenyl)-N-methylthiophen-3-amine) (5a);
obtaining a second intermediate (2-(4-(diethylamino)phenyl)-7-(4-methoxyphenyl)-4-methyl-6,7-dihydrothieno[3,2-b]pyridin-5(4H)-one) (6a) by quenching the first intermediate(5a) with water and extraction;
obtaining a third intermediate (6-bromo-2-(4-(diethylamino)phenyl)-7-(4-methoxyphenyl)-4-methylthieno[3,2-b]pyridin-5(4H)-one) (7a) by quenching the second intermediate (6a) with water and extraction;
obtaining a fourth intermediate (2-(4-(Diethylamino)phenyl)-7-(4-methoxyphenyl)-4-methylthieno[3,2-b]pyridin-5(4H)-one) (8a) by quenching the third intermediate (7a) with water and extraction;
obtaining KF (2-(4-(diethylamino)phenyl)-7-(4-hydroxyphenyl)-4-methylthieno[3,2-b]pyridin-5(4H)-one) by quenching the fourth intermediate(8a) with water and extraction; and
obtaining KF-DNBS (4-(2-(4-(diethylamino)phenyl)-4-methyl-5-oxo-4,5-dihydrothieno[3,2-b]pyridin-7-yl)phenyl 2,4-dinitrobenzenesulfonate) by quenching the KF with water and extraction.

3. The method of claim 2, wherein the step of obtaining the second intermediate (6a) is characterized by mixing and reacting the first intermediate (5a) with 4-methoxycinnamic acid, benzotriazol-1-yloxy)tris(dimethylamino)phosphonium hexafluorophosphate (BOP) and DIPEA.

4. The method of claim 2, wherein the step of obtaining the third intermediate (7a) comprises a step of mixing and reacting the second intermediate (6a) with N-bromosuccinimide.

5. The method of claim 2, wherein the step of obtaining the fourth intermediate (8a) comprises a step of quenching the third intermediate (7a), followed by reaction with an n-butyllithium solution.

6. The method of claim 2, wherein the step of obtaining the KF includes the steps of:
quenching the fourth intermediate (8a), followed by reaction with boron tribromide; and
quenching the resulting reaction mixture with water, followed by neutralization.

7. The method of claim 2, wherein the step of obtaining the KF-DNBS comprises a step of mixing and reacting KF with 2,4-dinitrobenzenesulfonyl chloride and triethylamine.

8. A composition for hydrogen sulfide detection, the composition comprising:
a probe for hydrogen sulfide (H₂S) detection, represented by the following Chemical Formula 1; and
2-formyl benzene boronic acid (2-FBBA) as a masking reagent: 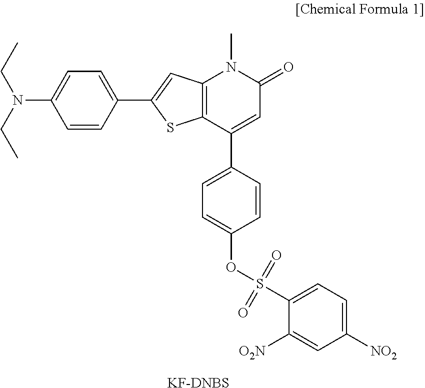 [SMILES: CCN(CC)c1ccc(-c2cc3c(s2)c(-c2ccc(OS(=O)(=O)c4ccc([N+](=O)[O-])cc4[N+](=O)[O-])cc2)cc(=O)n3C)cc1 ; "KF-DNBS"]
